(19) United States
(12) Patent Application Publication
(10) Pub. No.: US 2004/0175415 A1
(43) Pub. Date: Sep. 9, 2004

(54) Formulations and methods of delivery of intact tocopheryl succinate to humans

(76) Inventors: Alvin C. Chan, Ottawa (CA); Ching Kuang Chow, Lexington, KY (US)

Correspondence Address: FROMMER LAWRENCE & HAUG, 745 FIFTH AVENUE- 10TH FL., NEW YORK, NY, 10151, US

(21) Appl. No.: 10/379,644

(22) Filed: Mar. 5, 2003

(51) Int. Cl. A61K 9/70; A61K 31/355

(52) U.S. Cl. 424/449; 514/458

(57) ABSTRACT

Several formulations and methods of delivering anti-neoplastic tocopherol succinate to human subjects through transdermal and transmucosal routes to preserve its intactness are disclosed. Pharmaceutically acceptable carriers such as dimethyl sulfoxide, almond oil and water are used to process tocopherol succinate at different temperatures prior to applications. By administering tocopherol succinate in this manner, the molecular form of the tocopherol succinate is preserved, resulting in enhanced efficacy of the anti-neoplastic function in human. By using the formulations and delivery methods of the invention as sole treatment, several human cancer cases had been treated successfully in a relatively short period of time.

### FIELD OF THE INVENTION

This invention relates to novel formulations and methods of delivering tocopheryl succinate (hereinafter “TS”) to humans in order to preserve the intactness of the molecule. Appropriate combinations of TS and acceptable carriers such as dimethyl sulfoxide, almond oil, stearyl alcohol, petroleum jelly, mineral oil and distilled water; as well as novel differential temperature processing methods were developed to formulate TS. These formulations are successfully applied to humans via transdermal and transmucosal routes as evident by the appearance of circulating TS of the test subject.

### BACKGROUND OF THE INVENTION

### List of Prior Art Literatures

Barnett K T, Fokum F D, Malafa M P. Vitamin E succinate inhibits colon cancer liver metastases. J. Surg Res. 2002; 106(2):292-298.

Bos J D, Meinardi M M. The 500 Dalton rule for the skin penetration of chemical compounds and drugs. Exp Dermatol. 2000 Jun;9(3):165-9. Review.

Chan A C. Vitamin E and Arterioscleroses. Journal of Nutrition 1998 128: 1593-1596. Review

Chan AC et al. DRI report on vitamin C, vitamin E, selenium and carotinoids. National Academy Press, 2000, Institute of Medicine, US National Academy of Sciences, Washington D.C. pg1-506.

Chen G, Goeddel V. TNF-R1 signaling: A beautiful pathway. Science 2002 May; 296:1634-5.

Charpentier A, Groves S, Simmons-Menchaca M, Turley J, Zhao B, Sanders B, Kline K. RRR-alpha-tocopheryl succinate inhibits proliferation and enhances secretion of transforming growth factor-beta (TGF-beta) by human breast cancer cells. Nutr Cancer. 1993; 19(3):225-39.

Cheeseman K H, Holley A E, Kelly F J, Wasil M, Hughes L, Burton G. Biokinetics in humans of RRR-alpha-tocopherol: the free phenol, acetate ester, and succinate ester forms of vitamin E. Free Radic Biol Med. 1995 Nov;19(5):591-8.

Chow C K. Vitamin E and oxidative stress. Free Radical Biol. Med. 1991; 11:215-232. Review.

Chow C K. Vitamin E and cancer. In Nutrition and Disease update: Cancer (Kritchevsky, D and Carroll, K K ed.) AOCS Press, Champaign, IL; 1994, pp. 214-233. Review.

Demoler V, Isler O, Ringer B H, Salomom H, Karrer P. Organic synthesis of tocopheryl succinate Helv. Chim. Acta 1939, 22: 65-68.

Djuric Z, Heilbrun L K, Lababidi S, Everett-Bauer C K, Fariss M W. Growth inhibition of MCF-7 and MCF-10A human breast cells by alpha-tocopheryl hemisuccinate, cholesteryl hemisuccinate and their ether analogs. Cancer Lett. 1997 Jan 1;11 1(1-2):133-9.

ElAttar T M, Lin H S. Inhibition of human oral squamous carcinoma cell (SCC-25) proliferation by prostaglandin E2 and vitamin E succinate. J Oral Pathol Med. 1993 Oct;22(9):425-7.

Horwitt M K, Eliott W H, Kanjananggulpan P, Fitch C. Serum concentrations of alpha-tocopherol after ingestion of various vitamin E preparations. Am J Clin Nutr 1984, 40:240-245.

Hull W Heat-Enhanced Transdermal Delivery: A Survey Paper J. Appl. Res.-Clin. Expl. Therap. Vol 2, number 1, winter, 2002

Jha M N, Bedford J S, Cole W C, Edward-Prasad J, Prasad K N. Vitamin E (d-alpha-tocopheryl succinate) decreases mitotic accumulation in gamma-irradiated human tumor, but not in normal, cells. Nutr Cancer. 1999;35(2):189-94.

Kline K, Yu W, Sanders W. Vitamin E: mechanisms of action as tumor cell growth inhibitors. J Nutr. 2001 Jan;131(1):161S-163S

Lee V H. Mucosal drug delivery. J. Natl. Cancer Inst. Monogr. 2001, 29:41-44

Malafa M P, Neitzel L T. Vitamin E succinate promotes breast cancer tumor dormancy. J Surg Res. 2000 Sep; 93(1):163-70.

Malafa M P, Fokum F D, Mowlavi A, Abusief M, King M. Vitamin E inhibits melanoma growth in mice. Surgery. 2002 Jan;131(1):85-91

Neuzil J, Weber T, Schroder A, Lu M, Ostermann G, Gellert N, Mayne G C, Olejnicka B, Negre-Salvayre A, Sticha M, Coffey R J, Weber C. Induction of cancer cell apoptosis by alpha-tocopheryl succinate: molecular pathways and structural requirements. FASEB J. 2001 Feb; 15(2):403-15.

Neuzil J, Zhao M, Ostermann G, Sticha M, Gellert N, Weber C, Eaton J W, Brunk U T. Alpha-tocopheryl succinate, an agent with in vivo anti-tumour activity, induces apoptosis by causing lysosomal instability. Biochem J. 2002 Mar 15;362(Pt 3):709-15.

Prasad K N, Edwards-Prasad J. Effects of tocopherol (vitamin E) acid succinate on morphological alterations and growth inhibition in melanoma cells in culture. Cancer Res. 1982 Feb;42(2):550-5

Qian M, Kralova J, Yu W, Bose H R Jr, Dvorak M, Sanders B G, Kline K. c-Jun involvement in vitamin E succinate induced apoptosis of reticuloendotheliosis virus transformed avian lymphoid cells. Oncogene. 1997 Jul 10;15(2):223-30

Rama B N, Prasad K N. Study on the specificity of alpha-tocopheryl (vitamin E) acid succinate effects on melanoma, glioma and neuroblastoma cells in culture. Proc Soc Exp Biol Med. 1983 Nov;174(2):302-7.

Rimm E B, Stampfer M J, Ascherio A, Giovannucci E, Colditz G A, Willett W C. Vitamin E consumption and the risk of coronary heart disease in men. N Engl J Med. 1993 May 20; 328(20):1450-6

Rose A T, McFadden D W. Alpha-tocopherol succinate inhibits growth of gastric cancer cells in vitro. J Surg Res. 2001 Jan;95(1):19-22.

Salih H R, Starling G C, Knauff M, Llewellyn M B, Bavis P M, Pitts W J, Aruffo A, Kiener P A. Retinoic acid and vitamin E modulate expression and release of CD178 in carcinoma cells: consequences for induction of apoptosis in CD95-sensitive cells. Exp Cell Res. 2001 Nov 1;270(2):248-58.

Simmons-Menchaca M, Qian M, Yu W, Sanders B G, Kline K. RRR-alpha-tocopheryl succinate inhibits DNA synthesis and enhances the production and secretion of biologically active transforming growth factor-beta by avian retrovirus-transformed lymphoid cells. Nutr Cancer. 1995;24(2):171-85.

Slack R, Proulx P. Studies on the effects of vitamin E on neuroblastoma N1E 115. Nutr Cancer. 1989;12(1):75-82.

Stampfer M J, Hennekens C H, Manson J E, Colditz G A, Rosner B, Willett W C. Vitamin E consumption and the risk of coronary disease in women. N Engl J Med. 1993 May 20; 328(20):1444-9.

Turley J M, Sanders B G, Kline K. RRR-alpha-tocopheryl succinate modulation of human promyelocytic leukemia (HL-60) cell proliferation and differentiation. Nutr Cancer. 1992; 18(3):201-13

Turley J M, Fu T, Ruscetti F W, Mikovits J A, Bertolette D C 3rd, Birchenall-Roberts M C. Vitamin E succinate induces Fas-mediated apoptosis in estrogen receptor-negative human breast cancer cells. Cancer Res. 1997 Mar 1;57(5):881-90.

van Hoogdalem E, e Boer A G, Breimer D D. Paharmokinetics of rectal drug administration, Part I, General consideration and clinical applications of centrally acting drugs. Clin. Pharmacokinet 1991 21: 11-26.

Wajant H. The Fas signaling pathway: more than a paradigm. Science 2002, May 296: 1635-6

Weber T, Lu M, Andera L, Lahm H, Gellert N, Gariss M W, Korinek V, Sattler W, Ucker D S, Terman A, Schroder A, Erl W, Brunk U T, Coffey R J, Weber C, Neuzil J. Vitamin E succinate is a potent novel antineoplastic agent with high selectivity and cooperativity with tumor necrosis factor-related apoptosis-inducing ligand (Apo2 ligand) in vivo. Clin Cancer Res. 2002 Mar;8(3):863-9

Wu K, Shan Y J, Zhao Y, Yu J W, Liu B H. Inhibitory effects of RRR-alpha-tocopheryl succinate on benzo(a)pyrene (B(a)P)-induced forestomach carcinogenesis in female mice. World J Gastroenterol. 2001 Feb;7(1):60-5.

Wu K, Li Y, Zhao Y, Shan Y J, Xia W, Yu W P, Zhao L. Roles of Fas signaling pathway in vitamin E succinate-induced apoptosis in human gastric cancer SGC-7901 cells. World J Gastroenterol. 2002 Dec;8(6):982-6.

YuJ, Chien Y W. Pulmonary drug delivery: physiologic and mechanistic aspects. Crit. Rev. Ther Drug Carrier syst. 1997 14: 395-453.

Zhang Y, Ni J, Messing E M, Chang E, Yang C R, Yeh S. Vitamin E succinate inhibits the function of androgen receptor and the expression of prostate-specific antigen in prostate cancer cells. Proc Natl Acad Sci U S A. 2002 May 28;99(11):7408-13.

Tocopheryl succinate (“TS”) is a semi-synthetic product made from a simple chemical reaction using natural vitamin E (RRR-alpha-tocopherol) derived from soybeans and succinic anhydride. The method of its organic synthesis was reported over 60 years ago (Demole et al. 1939). Addition of the succinate moiety blocked the antioxidation functional group of the vitamin E molecule and renders the free tocopherol into a non-oxidizable form. This added feature has commercial benefit in prolonging the shelf life of TS when compared to free tocopherol. Thus, TS per se is not an antioxidant. For more than 30 years, TS has been available over the counter as one of many oral vitamin E supplements. Since TS remains as solid in room temperature, it is not as well accepted as a supplement when compared to other popular synthetic products such as tocopherol acetate, which is in oil form and resembles the appearance and property of natural vitamin E (tocopherol). Hence, limited market called for limited production of TS by industry. Presently, TS can be purchased over the counter of health food stores, labeled as ‘dry vitamin E’.

The trend of high vitamin E supplemental use by the general public over recent years is due to very strong epidemiological evidence indicting that vitamin E supplement can reduce the risk of heart and stroke events by 50 to 60% in man and women (Stampfer et al. 1993; Rimm et al. 1993). The mechanism by which vitamin E exerts protection against heart and stroke conditions by altering the eicosanoid profile in blood vessel cells and platelets has been systematically delineated and results reviewed by one of the joint inventors (Chan, 1998).

More than 20 years ago, Prasad first reported that TS has a unique anti-neoplastic activity for cancer cells when added directly into cell culture medium (Prasad and Edward-Prasad, 1982). Since then, this observation has been repeated, confirmed and extended to include over 90% of human cancer cell types by different laboratories. Among these mounting evidence of cell culture data came along a series of animal trials which confirmed the cell culture results that TS can indeed prevent transplanted cancer growth as well as chemically-induced cancer development in animals. While originally designed as oral supplement for vitamin E (tocopherol), TS exerts the unique cancer killing property that cannot be replaced by the free tocopherol (vitamin E). The fact that free vitamin E can prevent oxidative stress, but not cancer development or protection has been reviewed by one of the joint inventors (Chow, 1991; Chow, 1994).

Recently, TS has been demonstrated to effectively inhibit tumor development and progression in animals. In addition, precise and novel mechanisms by which TS induced tumor cell destruction have been clearly demonstrated by several different laboratories (see sections below for details and references).

However, when taken orally, TS is hydrolyzed to tocopherol (vitamin E) and succinic acid, a common metabolite found in our mitochondria during oxidation of glucose. Since effective killing of cancer cells by TS is totally dependent on the intactness of the TS molecule, it is necessary to develop other ways to administer TS so that its molecular intactness is not altered.

In U.S. Pat. No. 6,417,223, application Ser. Nos. 10/008,066 and 10/122,019, Sanders et al. disclosed tocopherols, tocotrienols, other chroman and side chain derivatives and their uses, including use as anti-neoplastic agent. Sanders et al. ignored the intactness of the TS molecule and includes oral among other routes of administrations. In fact, as of to date, there is no experimental data with regards to the formulation or human treatment methods provided. The present invention focuses on the formulations and processing of TS as well as the methods of successful administrations to humans.

Sanders et al. stated the applications to a vast majority of illnesses that include HIV and other viral diseases, all forms and types of human cancer, seven groups of human autoimmune diseases, and seven different types of skin disorders. Despite the vast range of diseases claimed, no human evidence was ever provided. On the other hand, the present inventors demonstrated the effectiveness of TS formulation in delivering intact TS into humans. By using these formulation and methods of administration, efficacious against several forms of human cancer has been demonstrated.

It is also noted that the major hypothesis relied on by Sanders et al. was with regards to induction of apoptosis by TS, an event that has now been proved to be secondary event which occur after lysosomal derangement and cell protein regulations (Zhang et al. 2002; Neuzil et al. 2002).

The present invention provides novel formulations and methods of transdermal and transmucosal delivery of TS to humans as evident by the appearance of TS in blood of test subjects. In addition, evidence of human cancers (basal cell skin cancer, recurring lymphoma, and others), which are successfully treated with TS delivered by these methods, is disclosed herein.

### SUMMARY OF THE INVENTION

It is an object of the present invention to teach the transdermal administration of TS to human cancer patients. According to one aspect of the invention, it provides a formulation containing an effective amount of tocopheryl succinate and pharmaceutically acceptable carriers suitable for transdermal administration to humans.

It is another object of the present invention to teach a TS formulation, which is effective and easy to prepare, and administered by the patients themselves.

According to another aspect of the invention, it provides a formulation containing an effective amount of tocopheryl succinate and pharmaceutically acceptable carriers suitable for transmucosal delivery to humans.

### BRIEF DESCRIPTION OF THE DRAWINGS

FIG. 1 shows the effect of TS administration by transdermal route plasma levels of TS.

FIG. 2 shows the effect of TS administration by transmucosal routes plasma levels of TS.

### DETAILED DESCRIPTION OF THE PREFERRED EMBODIMENT

TS was first reported to kill cancer cells in culture by Prasad more than 20 years ago (Prasad et al., 1982). Subsequently, the cancer killing effect of TS was confirmed by a vast majority of laboratories and this anti-neoplastic feature has extended to include over 90% of human cancer types as listed below:

breast (Carpentier et al. 1993; Djuric et al. 1997; Turlet et al. 1997)

colon (Barnett et al. 2002; Weber et al. 2002)

gastric (Rose et al. 2001; Wu et al. 2002)

leukemic (Turley et al. 1992)

lung (Jha et al. 1999; Salih et al. 2001)

lymphoid (Simmons-Menchaca et al. 1995; Qian et al. 1997)

melanoma (Prasad, 1982; Malafa et al. 2002)

neuroblastoma (Rama and Prasad 1983)

oral squamous (ElAttar and Lin 1993)

ovarian (Jha et al. 1999; Salih et al. 2001)

prostate (Israel et al. 2000; Zhang et al. 2002)

The study of cancer using classic approaches for small laboratory animals has been limited to several methods with their corresponding assumptions. These methods include the inoculation of cancer cells, cancer cell xenografts and chemically induced cancer development using known carcinogens. The efficacy of a drug is evaluated by its effectiveness in controlling or reducing cancer growth and development.

Using these approaches, TS has been shown by different laboratories to be an effective anti-neoplastic agent, which not only reduced tumor progression, but also prevents cancer formation induced by known carcinogens. Hence, TS has the features for an effective cancer prevention drug that has high potential as a chemotherapeutic agent, with no side effects. Results on the effectiveness of TS on cancer growth in animal studies reported recently are summarized in Table 1 below.

TABLE 1
In vivo effects of TS administration on cancer arrest in nude mice

| Tumor type/methods | TS dose | Outcome measurements |
|---|---|---|
| colon cancer cell | ip, 5 injections | tumor volume reduced |
| xenografts | of 10 mg/day for | by TS treatment (p < 0.01) |
| (Weber et al 2002) | 10 days | |
| murine melanoma | ip, 15 injections | Treatment resulted in reduction of |
| inoculations | at 3 mg/mouse | tumor volume (p < 0.01) and |
| (Malafa et al. 2002) | | proliferation. Also increases in |
| | | apoptosis |
| (benzopyrene) chemical- | ip, 8 injections | ip treatments resulted in 84% |
| induced forestomach | at 20 mg/kg | reduction in tumor volume & 50% |
| cancer(Wu et al. 2001) | intragastric 16 | less tumor incident. |
| | times at either | 1.25 g/kg or 2.5 g/kg gastric TS gave |
| | 1.25 g/kg or | 63% and 81% reduction in tumor |
| | 2.5 g/kg | volume and 18 and 23% reduction in tumor |
| | | incident. |
| colon cancer | ip, 4 injections | treatment resulted in significant |
| xenografts | at 5 mg once | 75% reduction in tumor volume |
| (Neuzil et al. 2001) | every 3 days | and increase apoptotic cells. |
| human breast | ip, 7 injections | no effect with sc injections. |
| cancer cells | at 3 mg/day or | ip injection resulted in 63% |
| inoculation | sc, at 150 mg/kg | reduction in tumor weight. |
| (Malafa et al. 2000) | per day | ip injection also reduce |
| | | angiogensis via the suppressing |
| | | the expression of VEGF (vascular |
| | | endothelial growth factor) |

During the past few years, major breakthrough in TS research has clearly delineated the mechanism by which TS causes cancer cell death without affecting normal cells. When given as intact molecule, TS exerts its anti-neoplastic effect by several current hypothesis of the apoptosis pathway. These include intra- and inter-cellular signaling pathways: (1) the TGF-Beta (transformed growth factor-beta) pathway; (2) the G protein kinases pathways consist of c-Jun N-terminal kinase (JNK) and mitogen-activated protein kinase (MAPK) pathways; and (3) the FAS (CD95/APO-1) signaling pathway (Chen and Goeddel, 2002; Waljant, 2002).

While this specific apoptotic function of TS has been reviewed (Kline et al. 2001), recent discoveries offered alternate novel mechanisms by which TS kills cancer cells before the occurrence of apoptosis. For example, Neuzil et al. (2002) demonstrated that TS caused major disruption of cellular lysosomal prior to the induction of apoptosis in several lines of cancer cells in culture, indicating that apoptosis is secondary to lysosomal disruption.

In addition, Zhang et al. (2002) first reported that in human prostate cancer cells, TS inhibits the expression of the androgen receptors by means of transcriptional and post-transcriptional modifications of the androgen receptor protein. More importantly, TS strongly suppresses the expression of PSA (prostate specific antigen), a functional clinical detector molecule for the diagnosis of human prostate cancer. Therefore, it is clear that TS is able to regulate the expression of significant cellular proteins upward or downward, causing major membrane disruption before the occurrence of apoptosis. Hence TS is a multi-functional molecule involved in at least several novel mechanisms in exerting its caner killing effects.

In human, oral ingestion of TS was shown to convert to tocopherol due to extreme high level of esterase in the digestive juice (Howritt et al. 1984, Chessman et al. 1995). Therefore, in order to maintain the intactness of the TS molecule, alternate routes of administration must be developed to ensure that TS is delivered per se in vivo.

The present inventors sought to identify appropriate carriers and formulation mix for TS that are safe and effective for human transdermal as well as transmucosal deliveries.

According to the reference of Chemistry (the Merck index), TS is not readily soluble in vegetable oils. After many trials and errors, and with the combination of heat as a variable during processing, the present inventors found appropriate formulating ratios of TS to dimethyl sulfoxide, to almond oil, to stearyl alcohol, petroleum jelly, mineral oil and to water, that can be effective and safe to be used in humans. From these formulations, the inventors developed a serious of gel and solid with TS that can be used for transdermal and transmucosal deliveries.

Transdermal Delivery of TS

The skin is the largest organ in human and it forms a natural barrier between the environment and the human body. Thickness of skin differs a great deal according to sites and locations. It is composed of three layers: the epidermis, the dermis and the subcutaneous fatty tissue. The epidermis ranges from 0.15 to 0.80 mm in thickness and is the outermost part, called the stratum corneum or horny layer. This layer consists of several layers of flattened dehydrated dead cells. The dermis is 3-5 mm thick and is consisted of non-cellular tissues (collagen and other structural proteins). The dermis is rich in small blood and lymphatic vessels, nerves endings, hair follicles and sebaceous and sweat glands.

Penetration of drugs from the outside through the layers below the skin and their entrance into blood capillaries and vessels is called percutaneous (also known as transdermal delivery). Transdermal delivery of drugs has several significant advantages over that of oral or injection. Firstly, skin drug delivery avoids gastrointestinal hydrolysis and metabolism of drugs by intestinal mucosa cells. This route also avoids first-pass drug deactivation by the liver that occurs during oral intake of drugs, and therefore, extending the intactness and activity of drug given. More importantly, it allows multiple applications with little side effects often encountered with oral and injectable forms of drug administrations. In terms of health cost, there is a significant reduction of savings from incurring cost of technicians or nurses who are required for injectable drugs, because it can be self-administrated.

Conversely, there are limitations by which drugs are delivered by the transdermal route. Molecules such as insulin cannot be given this way due to the large molecule size. Also its hydrophilic nature renders it difficult to cross the membrane barriers. The charge of the molecule is also important in transdermal delivery of drugs; for example, a charged molecule is far more difficult getting in than a non-charged molecule by transdermal means of administrations.

Factors affecting transdermal drugs delivery include:

Chemical nature of the drug

the smaller MW, better penetration, compounds with a molecular weight around 500 (MW of TS is 530) or lower, can be delivered with zero order kinetics (Bos, 2000)

more hydrophilic (oil-soluble), better penetration

non-ionic in nature (no charge), better penetration than charged molecule

External factors affecting drug delivery

heat: heat is known to increase skin permeability, blood vessel wall permeability, rate-limiting membrane permeability as well as drug solubility in formulation

both the drug/carrier as well as skin temperature highly influence the rate and amount of drug delivered. For example, it has been reported that a 5-degree increase in skin temperature caused a 2 to 5 fold increase in drug delivered

when given at a room with temperature raised to 40° C., a 3-fold increase in dermal crossing of salicylate was found in man (Hull, 2002)

raising the skin temperature by direct infrared heating element has been shown to cause a 2-3 fold increase in delivered drug (Hull, 2002)

Specific Formulations of TS and Methods of Transdermal Administration to Human

TS is a small lipid soluble molecule with molecular weight of 530. It has a melting point of 72° C. and it stays as solid in room temperature. It is heat stable and resistant to oxidation due to the free OH group of the molecule is blocked by condensation reaction with succinic anhydride. Hence, TS is not an antioxidant. The formulations of the present invention involve using heat in processing method and the appropriate range ratios of TS:dimethyl sulfoxide:almond oil:stearyl alcohol:petroleum jelly:mineral oil:distilled water (1:0.01 to 0.4:0.05 to 0.4:0 to 0.1:0 to 0.1:0 to 0.1:0.01 to 0.05 respectively). This formulated TS mix is applied to a heated skin area of 15×15 cm using slow message motion until all TS preparation is used up and absorbed by skin area (between 5-10 minutes of message time). Area of skin selected should be the most proximal to the tumor site and thick skin areas such as the sole, knee, palm and areas covered with hair should be avoided. Examples for different formulation ratios for transdermal administration for TS are shown below (Table 2). The present inventors discovered that the most effective amount of TS is between the ranges of 400 to 1200 mg.

TABLE 2
Examples of six formulations for transdermal administration

| Ingredients | #1 | #2 | #3 | #4 | #5 | #6 |
|---|---|---|---|---|---|---|
| Tocopheryl succinate | 58.7* | 51.3 | 52.6 | 48.1 | 46.0 | 60.5 |
| Dimethyl sulfoxide | 11.6 | 20.5 | 15.9 | 19.2 | 28.0 | 21.2 |
| Almond oil | 17.3 | 10.3 | 21.1 | 19.2 | 13.0 | 8.9 |
| Stearyl alcohol | 2.9 | 5.1 | 1.6 | 2.9 | 5.0 | 3.5 |
| Petroleum jelly | 2.9 | 5.1 | 2.1 | 3.8 | 1.0 | 1.4 |
| Mineral oil | 1.7 | 5.1 | 3.2 | 1.9 | 2.7 | 1.6 |
| Distilled water | 5.8 | 2.6 | 3.7 | 4.8 | 4.3 | 2.9 |

*Percentile or ratio in weight or volume.

Calculation of TS Dose and Relationship with Blood Level

Evidence from cell culture and animal studies revealed that the effective concentration of TS in destroying cancer cells ranges from 25 to 50 micromolar (Zhang et al. 2002). Assuming that the blood volume of an adult individual is 6 liters and about half of TS administered is transferred from skin microvessels to blood, the amount of TS estimated by theoretical calculation required to achieve this level is around 250 mg of TS.

Methods of Application of TS Formulation

The TS formulation can be used at a rate of 2-3 times a day, with 250 mg of TS for each administration to achieve the desirable level.

Selection of Skin Areas

the thinner the skin, the better transport—use skin area that is not thick (avoid sole, palm, knee and area with hair); the thinnest skin is near the human private part and behind the ears

the closer to the site of tumor, the better

an area of about 1 square foot is the upper limit for area; use the same site for all treatments

Preparation of the TS Formulation and Skin before Application

it is best to start in the morning after bath or shower

warm up selected skin site with a heated beanbag or other heating device, until skin is too hot for you to stand

gently rub in the TS formulation with fingers within selected skin zone and message it in with slow but forceful motions

cover site with dry cloth and reapply beanbag to keep the temperature of skin site warm

Transmucosal Delivery of TS

The mucosal are specialized epithelial cells that line up different orifice of the human body and they include oral mucosa (buccal, sublingual and gingival mucosa), nasal mucosa, pulmonary mucosa, rectal mucosa and vaginal mucosa. These sites are rich in small blood vessels and are known targets for drug delivery (for reviews, see van Hoogdalem 1991, Yu and Chien 1997, Lee 2001 and www.nlm.nih.gov/medlineplus). The mucosa route of drug administration provides direct entry of drug into the systemic circulation thus avoiding the hepatic first-pass metabolism and degradation by gut enzymes. It has distinct advantage for patient who cannot tolerate oral or iv delivered drugs. In this case, intact absorption of TS by these routes is possible due to lack of digestive enzymes that hydrolyzed TS when taken orally. In addition, similar to the transdermal route, drugs given this way will escape immediate hepatic metabolism and hence rendering a longer half-life. However, there are limitations of such an approach and they resemble those disclosed under transdermal applications herein.

Formulation and Application of TS for Transmucosal Delivery by Rectal and Vaginal Routes

TS is a low molecular weight lipid soluble compound that remains as solid in room as well as body temperatures. In order to fulfill the requirement of a liquid/gel form suitable for transmucosal delivery, it is necessary to develop a formulation so that the preparation can remain solid at room temperature but change to liquid state upon insertion to the human body. The present inventors developed such a formulation by using the appropriate proportion of TS to dimethyl sulfoxide to water to almond oil. This mixture has the range of ratios of TS:dimethyl sulfoxide:almond oil:stearyl alcohol:distilled water (1:0.1 to 0.6:0.05 to 0.2:0 to 0.1:0 to 0.15, respectively). Differential temperatures and centrifugation were used during processing in order to transform TS from solid state to gel state and reversing it to solid state again. The final product with a bullet shape was formed after centrifugation at 4 degree C. It remains as solid in room temperature but once inserted to the human orifice, it melts at 36 degree C. Examples for different formulation ratios for TS administration via transmucosal delivery are shown in Table 3. Processing temperature is changed from room temperature to 75 degree C., and then change to 4 degree C again with the aid of centrifugation. The present inventors discovered that the most effective amount of TS is between the ranges of 250 to 1000 mg.

TABLE 3
Examples of six formulations for transmucosal delivery

| Ingredients | #1 | #2 | #3 | #4 | #5 | #6 |
|---|---|---|---|---|---|---|
| Tocopheryl succinate | 50.0* | 58.5 | 44.6 | 62.5 | 43.8 | 52.5 |
| Dimethyl sulfoxide | 30.0 | 29.2 | 36.6 | 26.1 | 38.8 | 33.5 |
| Almond oil | 10.0 | 5.9 | 7.8 | 6.8 | 9.4 | 7.6 |
| Stearyl alcohol | 5.0 | 3.5 | 5.8 | 2.6 | 3.9 | 4.2 |
| Distilled water | 5.0 | 2.9 | 5.2 | 2.0 | 4.1 | 2.2 |

*Percentile or ratio in weight or volume.

Calculation of Dose for Transmucosal TS Delivery

In general, drugs delivered by mucosal routes have a range of crossing into the human body at 30 to 50% of the dose given. The calculation of effective dose is similar to that developed for transdermal route disclosed herein. Therefore, to achieve a constant 25micromolar concentration of TS in blood, 250 mg TS will be administered twice a day. Study with male and female human subjects via rectal and vaginal delivery are described below which demonstrated successful delivery of intact TS via the rectal and vaginal routes.

Toxicity Concerns

Judging from the number of human subjects reported in this study, there is no concern regarding the safety of this novel treatment procedure. The hydrolyzed products of TS are tocopherol (vitamin E) and succinic acid, an endogenous metabolite produced during the oxidation of carbohydrates, amino acids and fat. The upper safety limit for vitamin E is set at 1000 mg or 1 gm by the most recent issue of DRI (Dietary Reference Intake) for vitamin C, vitamin E selenium and carotenoids. The DRI report was published in 2000 and one of the joint inventors of the present invention served on the committee from which the report was sponsored by the Institute of Medicine, US National Academy of Sciences (Chan et al. 2000). The upper dose limit claimed in the invention herein, is below or around the upper safety limit set at 1 gm by the Institute of Medicine for oral ingestion.

Human Studies: Uptake of TS into Blood by Transdermal Administration

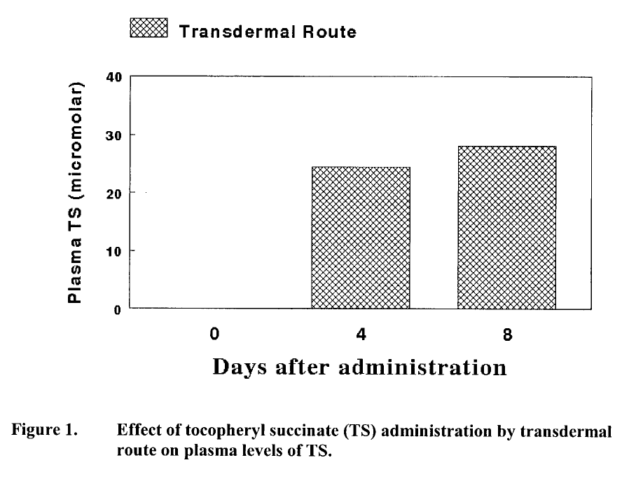

A male subject, age 53 was given this TS formulation via dermal means at a dose of 300 mg three times a day spread under 6 hours intervals for a period of 7 days. Blood was collected at day 0, day 4 and day 8. Detailed protocol is shown in Table 4 below. Plasma was denatured in 2 volume of ethanol. Samples were stored frozen pending for analysis. Following extraction, levels of TS were determined by HPLC method equipped with UV and fluoresce detectors (Slack et al. 1989). Levels of TS in plasma were 0, 24.5 and 28.1 micromolar (micromole/liter) for days 0, 4 and 8 after transdermal treatment. As expected, level of TS was undetectable in plasma of two control subjects that were not treated with TS. Since TS is a semi-synthetic compound, it will not be detected in individual who does not consume it. Results from this study are shown in FIG. 1.

TABLE 4
Experimental protocol for the study of
transdermal delivery of TS in humans

| Day of treatment | Time of treatment | Dose given | Blood drawn |
|---|---|---|---|
| 0 | — | 500 mg | 8 pm |
| 1 | 11:30 am; 10:15 pm | 500 mg | — |
| 2 | 11:05 am; 11:05 pm | 500 mg | — |
| 3 | 12:45 pm; 11:00 pm | 500 mg | — |
| 4 | 12:50 pm; 11:00 pm | 500 mg | 8 pm |
| 5 | 11:00 am; 11:00 pm | 500 mg | — |
| 6 | 10:50 am; 11:00 pm | 500 mg | — |
| 7 | 11:00 am; 11:00 pm | 500 mg | — |
| 8 | — | — | 9 am |

Uptake of TS into Blood by Transmucosal Administration

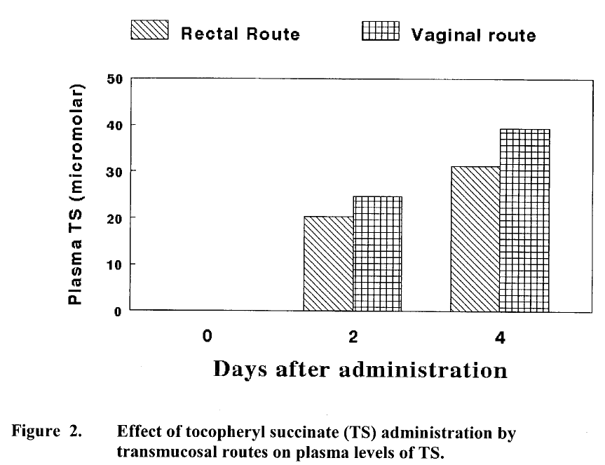

A male and female subject was each given formulated TS in suppository form containing 250 mg of TS. They were instructed to insert it in the morning after bowel movement and shower, and repeat it before bedtime. Blood was collected on day 0, day 2 and day 4. Plasma was separated from blood and TS was determined as described above. Results showed undetectable level of TS on day 0 of the study. Levels of TS in plasma ranged from 20 to 40-micromolar after 2 and 4 days of transmucosal administration. Result from this study is shown in FIG. 2.

Case Reports on use of TS Formulation

Case 1 (Recurring Lymphoma)

A 80-year-old male was found to have lymphoma in 2001, with lumps protruding from the neck region. Visits to oncologist resulted in chemotherapy treatments (3-5 days rounds 3 times with resting periods between treatments). Lumps were found to regress after chemotherapy. Nine months later, the lymphoma recurred with new lump in the neck measured (7.5×1.23×4.2 cm). Patient started using the TS formulation around the neck region at a dose of 0.8 gm twice a day, and three weeks into treatment, tumor started to regress. After eight weeks into treatment, tumor size was reduced by half (measured 3 cm in length). Complete regression of tumor occurred 12 weeks after treatment.

Case 2 (Prostate Cancer, Spread)

A 50-year-old male has metastasized prostate cancer in liver and bone. There is a lot of pain and patient is under prophylactic chemotherapy once a month. Patient started to use the TS formulation at a dose of 0.4 gm three times a day at the end of November, 2002. While there is no end point measurement made to determine tumors regression, two weeks into treatment, patient felt pain and discomfort level were reduced to more than half. With this improvement, patient opts to continue to use the TS formulation and benefits continue.

Case 3 (Recurring Sarcoma)

A 52-year-old male had recurring sarcoma on mid-penis in November 2002. Since this tumor is known not respond well to radiation and chemotherapy, doctor advised removal of organ to which the patient refused. After using the TS formulation for 4 weeks, tumor size was reduced by 30%. Patient continues to use formulation.

Case 4 (Nasopharyngeal Cancer)

A 53-year-old female was found to have nasopharyngeal cancer (stage 4) in August, 2002. She went through radiotherapy and some limited chemotherapy because the patient has hepatitis B. The TS formulation was used 5 days before treatments and continued through out all treatments at a dose of 0.5 gm twice a day. Despite of the stage 4 diagnoses, tumor regression was impressive and patient returned to work in February, 2003.

Case 5 (Basel Cell Skin Cancer)

A 73-year-old male has basal cell skin cancer for 20 years. The cancer recurs every year and treatment consisted of burning of skin tumor with either liquid nitrogen or dry ice. In September, 2002, several lumps appeared on his face, and they were painful and sensitive to touch. He started using the TS formulation in late September, 2002 as topical lotion. Partial recovery was detected after 3 weeks of continuous use. All spots were proclaimed clear by the patient after 8 weeks of treatment period.

Usefulness of the Application

The invention described herein can be used as an anti-cancer drug alone or as an adjunct for cancer treatment for over 90% of human cancer. It is very easy to administer (self or by another) and therefore has a superior saving value in cutting down the cost of technicians and nurses for injectable forms of drug delivery. It will also further cut cost in reducing the duration of hospital stay often associated with chemotherapy treatment. This low cost feature is especially in demand from developing countries or the third world where a lack of cancer treatment facility and cost may render most of the patients untreated. Most importantly, these effective formulations and delivery methods are totally non-invasive and non-toxic.

Due to the lack of side effects and toxicity, much of the suffering caused by conventional chemo- and radiotherapy on cancer patients can be eliminated or significantly reduced. In the context of market development, products can be used for cancer patients, or for cancer survivors who fear the recurrence of cancer. It also has a market for the normal or healthy population who may opt to use the product periodically for prophylactic purposes.

It is to be understood that the embodiments and variations shown and described herein are merely illustrative of the principles of this invention and that various modifications may be implemented by those skilled in the art without departing from the scope and spirit of the invention.

We claim:

1. A formulation containing an effective amount of tocopheryl succinate and pharmaceutically acceptable carriers suitable for transdermal administration to humans.

2. The formulation of claim 1, said effective amount of tocopheryl succinate is 400 to 1200 mg.

3. The formulation of claim 1, said pharmaceutically acceptable carriers are dimethyl sulfoxide, almond oil, stearyl alcohol, petroleum jelly, mineral oil and distilled water.

4. The formulation of claim 1, wherein said tocopheryl succinate is mixed with the pharmaceutically acceptable carriers in the ratio of tocopheryl succinate: dimethyl sulfoxide: almond oil: stearyl alcohol: petroleum jelly: mineral oil: distilled water 1:0.01 to 0.4:0.05 to 0.4:0 to 0.1:0 to 0.1:0 to 0.1:0.01 to 0.05 respectively.

5. A formulation containing an effective amount of tocopheryl succinate and pharmaceutically acceptable carriers suitable for transmucosal delivery to humans.

6. The formulation of claim 5, said effective amount of tocopheryl succinate is 250 to 1000 mg.

7. The formulation of claim 5, said pharmaceutically acceptable carriers are dimethyl sulfoxide, almond oil, stearyl alcohol and distilled water.

8. The formulation of claim 5, wherein said tocopheryl succinate is mixed with the pharmaceutically acceptable carriers in the ratio of tocopheryl succinate: dimethyl sulfoxide: almond oil: stearyl alcohol: distilled water 1:0.1 to 0.6:0.05 to 0.2:0 to 0.1:0 to 0.15 respectively.

9. The formulation of claim 5, further involving differential temperatures and centrifugations during processing to enable the finished product to stay as solid in room temperature but to transform into liquid/gel at 36 degree C. at the optimal body temperature.

10. A transdermal delivery method of administering the formulation of claim 1 to human subject, comprising warming an appropriate area of the skin wherein said formulation will be applied and applying the formulation onto the warmed skin area using slow message motion until all has been absorbed by the skin area.

11. The transdermal delivery method of claim 10, further comprising the step of covering the skin area warm for short period of time after the transdermal application.

12. A transmucosal rectal and virginal delivery method to deliver the formulation of claim 5 to human subject, comprising applying the formulation to rectal and vaginal walls to exert an efficacious anti-neoplastic function suitable for cancer of the colon, rectum and uterus and preserves the intact properties of said tocopheryl succinate.

13. A transmucosal rectal and virginal delivery method to deliver the formulation of claim 9 to human subject, comprising applying the formulation to rectal and vaginal walls to exert an efficacious anti-neoplastic function suitable for cancer of the colon, rectum and uterus and preserves the intact properties of said tocopheryl succinate.
